(19) United States
(12) Patent Application Publication
(10) Pub. No.: US 2016/0338823 A1
(43) Pub. Date: Nov. 24, 2016

(54) JUXTARENAL STENT AND METHODS

(71) Applicant: Indiana University Research and Technology Corporation, Indianapolis, IN (US)

(72) Inventor: A. George Akingba, Carmel, IN (US)

(73) Assignee: Indiana University Research and Technology Corporation, Indianapolis, IN (US)

(21) Appl. No.: 15/158,325

(22) Filed: May 18, 2016

Related U.S. Application Data
(60) Provisional application No. 62/163,175, filed on May 18, 2015.

(51) Int. Cl. A61F 2/07; A61F 2/86; A61F 2/954; A61F 2/844

(52) U.S. Cl. CPC A61F 2/07; A61F 2/844; A61F 2/86; A61F 2/954; A61F 2002/8483

(57) ABSTRACT

Stents including a cylindrical body having a distal portion, a skirt having an apex coupled to the distal portion, and an extension mechanism adapted to expand the skirt are disclosed. Methods of inserting such stents are also disclosed.

### CROSS-REFERENCE TO RELATED APPLICATIONS

This application claims priority to U.S. Provisional Application Ser. No. 62/163,175, filed on May 18, 2015, the entire disclosure of which is hereby expressly incorporated by reference.

### FIELD OF THE DISCLOSURE

This disclosure relates stents and methods of inserting stents into the human body. More specifically, this disclosure relates to juxta-renal aneurysms and stents capable of repairing these aneurysms and methods of inserting stents to repair aneurysms.

### BACKGROUND

Aneurysms are localized, blood-filled balloon-like bulges in the wall of blood vessels. Aortic aneurysms are enlargement/dilation of the aorta and often go unnoticed. It is often during a medical procedure that the aortic aneurysm is discovered. Aortic aneurysms can cause weakness in the wall of the aorta and, if left untreated, can rupture causing massive internal bleeding, which typically results in death.

Stents allow for medical doctors to repair aortic aneurysms by reinforcing the aortic wall. However, many conventional stents are straight and, thus, require doctors to attach a straight stent to healthy parts of various arteries.

To combat this problem, doctors have used various tubular modular stents/stent systems, such as endovascular aneurysm repair (or endovascular aortic repair) (EVAR). However, techniques using such stents cannot be used when aneurysms extend to or involve the renal arteries, because there is no healthy tissue to which the stents may be attached (e.g., when the aneurysm has substantially eliminated any neck area posterior to the renal arteries).

While custom stents (endografts) can be created, such stents may take weeks to months to manufacture and, thus, are not suited for emergencies, such as when an aneurysm is discovered during surgery.

A need therefore exists to address issues of endovascular aneurysm repair, where there is a lack of sufficient healthy tissue to which to attach a conventional stent in emergency situations.

### SUMMARY

The present disclosure provides stents capable of being deployed in an emergency situation and attain wall apposition at diseased areas. In some embodiments, stents may include a cylindrical body having a distal portion, a skirt having an apex in mechanical communication with the distal portion, and an extension mechanism adapted to expand the skirt.

In various embodiments, the extension mechanism may be inflatable. Also, in some embodiments, the inflatable extension mechanism comprises a plurality of inflatable chambers, which may be interconnected. Furthermore, in some embodiments, the plurality of inflatable chambers may be interconnected with intermediary chambers comprising a one-way valve.

In yet additional embodiments, the extension mechanism may be slidable along the cylindrical body, which may be configured to lock in a deployed position. The extension mechanism may also configured to unlock from the deployed position. Accordingly, the stent may include a detent in mechanical communication with the cylindrical body. In some embodiments, the stent may comprises a plurality of struts pivotally connected to the skirt and a rib.

Accordingly, various stents disclosed herein may include a cylindrical body having a distal portion, a skirt having an apex coupled to the distal portion, and means for expanding the skirt. In some embodiments, the expanding means includes an inflatable member. In some embodiments, the expanding means includes a plurality of struts pivotally engaged with the skirt and an extension mechanism.

In some embodiments, the skirt may comprises a wire support. Also, in some embodiments, the skirt may be pleated. To aid in adhesion to the wall of a blood vessels, stents may have a skirt that includes a plurality of barbs.

Methods of implanting a stent into a human body may comprise sliding a stent into a blood vessel along a guide wire, the stent comprising a cylindrical body having a distal portion, a skirt having an apex coupled to the distal portion, and an extension mechanism adapted to expand the skirt, and expanding the skirt to secure the stent to the blood vessel. In various embodiments, expanding the skirt may include inflating the extension mechanism and/or may include sliding the extension mechanism along the cylindrical body. Various methods may also include locking the stent in a deployed configuration.

Furthermore, as the present disclosure provides stents capable of being deployed in emergency situations, such stents are also capable of being deployed in non-emergency situations and, thus, makes them a suitable replacement for conventional stents.

### BRIEF DESCRIPTION OF THE DRAWINGS

The above mentioned and other features and objects of this disclosure, and the manner of attaining them, will become more apparent and the disclosure itself will be better understood by reference to the following description of exemplary embodiments of the disclosure taken in conjunction with the accompanying drawings, wherein:

FIG. 1 illustrates a frontal view of a stent according to various embodiments;

FIGS. 2A-C are various cross-sectional views of exemplary stents in the un-extended position;

FIG. 3A-C are an illustrative views of exemplary stents in the deployed position;

FIG. 4 illustrates an un-extended stent inserted near an aneurysm; and

FIG. 5A-B illustrate deployed stents inserted near an aneurysm according to various embodiments;

FIG. 6 is a cross-sectional view of a deployed stent inserted near an aneurysm according to various embodiments; and

FIG. 7 is a cross-sectional view of a plurality of deployed stents according to various embodiments.

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplification set out herein illustrates exemplary embodiments of the disclosure, in various forms, and such exemplifications are not to be construed as limiting the scope of the disclosure in any manner.

### DETAILED DESCRIPTION

The embodiments disclosed below are not intended to be exhaustive or limit the disclosure to the precise form disclosed in the following detailed description. Rather, the embodiments are chosen and described so that others skilled in the art may utilize its teachings.

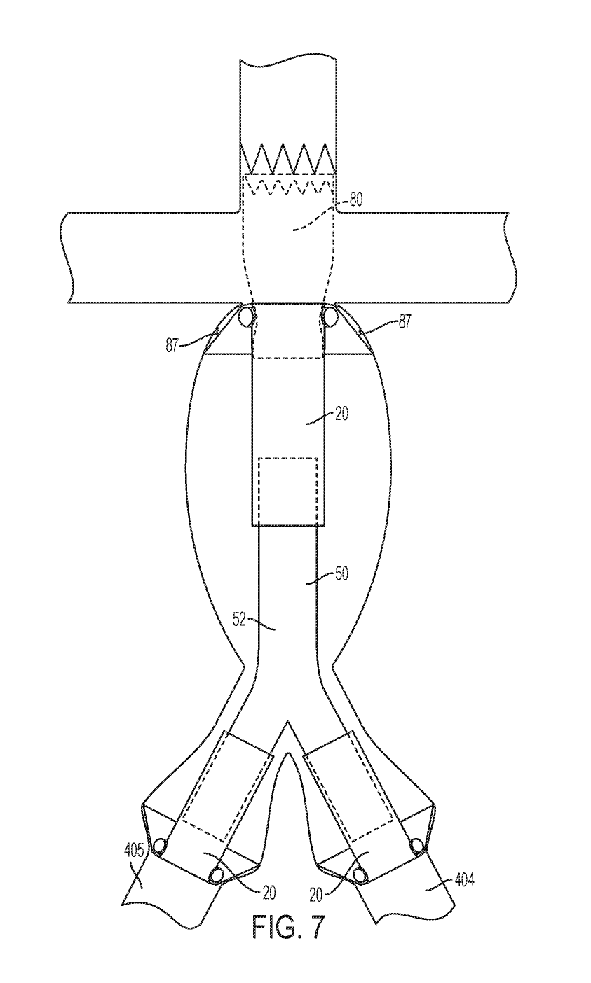

As used herein, any use of the terms “distal” and “proximal” or similar terms refers to the stent and not to any position within a body (e.g., distal or proximal of the heart). Accordingly, one of skill in the art would appreciate that the various stents disclosed herein may be utilized such that the “distal” portion of the cylindrical body may be configured distally or proximate to the heart, for example, as exemplified in FIG. 7. In other words, the word “distal” or “proximal” is independent of the stents configuration in a body.

The present disclosure provides a stent having a cylindrical body having a distal portion, a skirt having an apex in mechanical communication with the distal portion, and an extension mechanism adapted to expand the skirt.

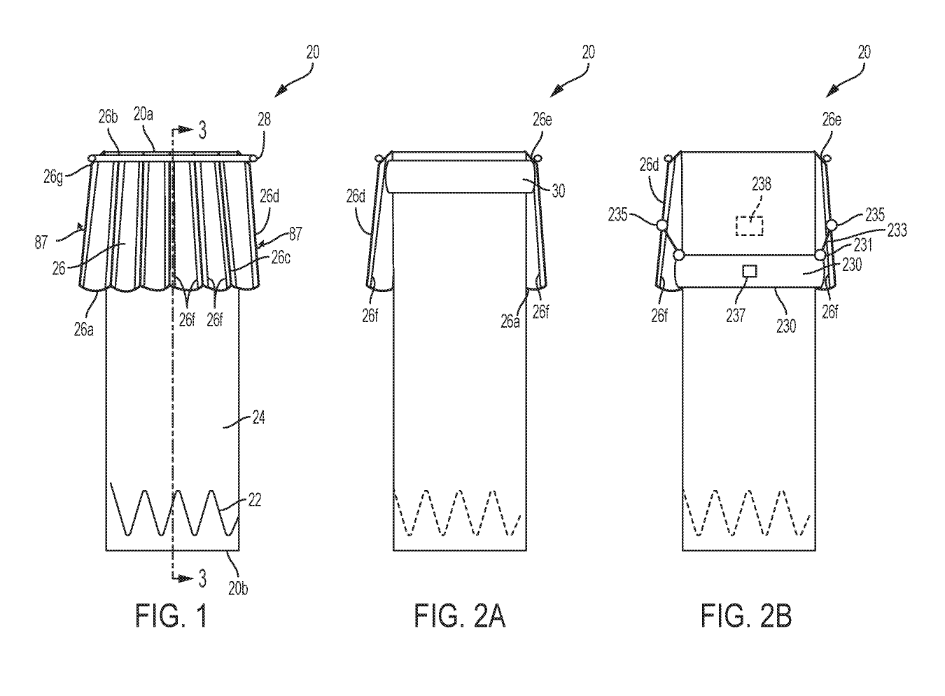

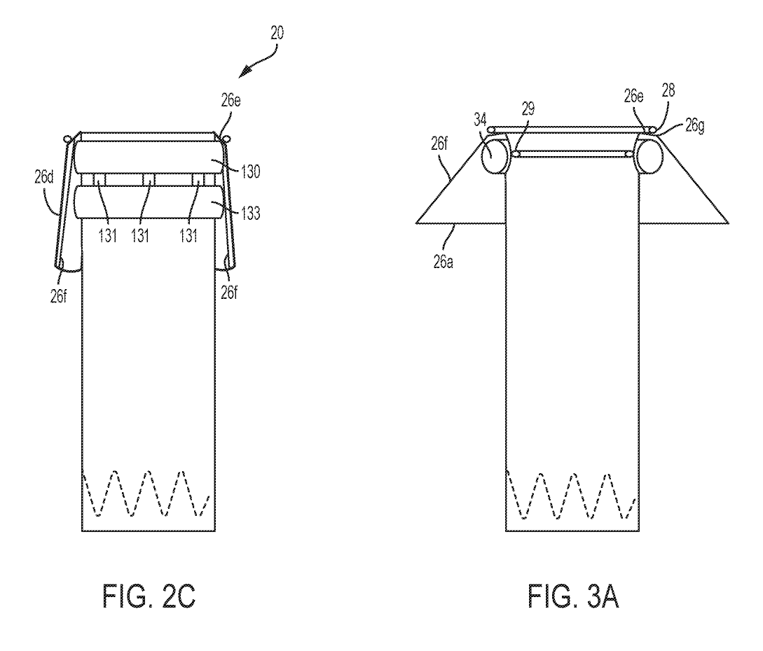

FIGS. 1, 2A-C, and 3A-C show frontal and cutaway drawings of an exemplary stent 20 according to various embodiments. FIG. 1 is an external side elevational view of the exemplary stent. Stent 20 includes a cylindrical body having a distal end 20a. In various embodiments, the cylindrical body may be comprised of a wire stent matrix 22 attached to graft material 24 and extending from a proximal end 20b to distal end 20a. For the sake of simplicity, only a small portion of the wire stent matrix 22 is shown. It is understood that the graft material 24 can be on either or both of the interior and exterior of the stent, as required for proper structural and functional properties, including the creation of sealing surfaces between the stent and a lumen of the body.

Stent 20 may include a skirt 26 having an apex in mechanical communication with the distal end 20a, for example, skirt 26 may be attached proximate to the distal end 20a. Skirt 26 has an opened end 26a that is spaced apart from an attached end 26b coupled to the body of stent 20 at the distal end 20a. In some embodiments, skirt 26 may include a plurality of pleats 26c that extend circumferentially around the stent body. In some embodiments, each pleat 26c includes a pair of wire supports 26f that extend along the axial length of skirt 26. As one example, each wire support 26f is attached by stitches along its length to graft material 24. In some embodiments, stent wire supports 26f are located on the interior surface of the pleat material.

In some embodiments, the immediately adjacent wire supports 26f of two adjacent pleats 26c contain between them a folded amount of graft material 24. This folded portion of graft material is narrow near the distal end 20a of skirt 26, and increases to a maximum width at the proximal end of skirt 26 (i.e., such as a generally triangular shape with the apex of the pleat material being located near distal end 20a).

Some embodiments of stent 20 further include a circular support wire 28 that extends circumferentially around the distal end of skirt 26. As shown in FIG. 1, in some embodiments, support wire 28 is placed on the exterior of skirt 26. However, in yet other embodiments, support wire 28 may be located along the inner surface of skirt graft material 24 (e.g., in between the skirt and the cylindrical body of stent 20).

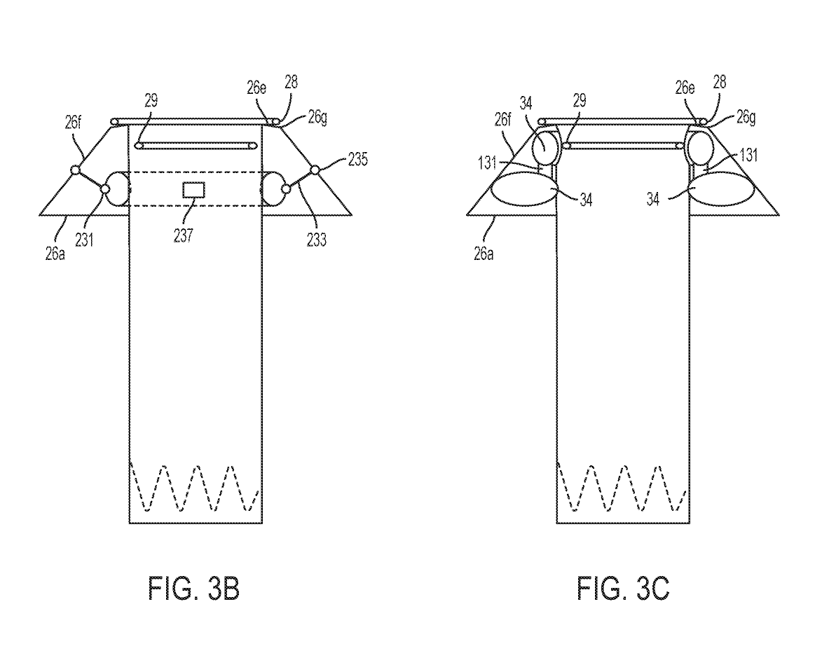

Some embodiments of skirt 26 include a shoulder segment 26e located between the open distal end of stent 20 and a corner of pleat 26c. As seen in FIGS. 2, and 3, shoulder segment 26e and the outer surface 26d of skirt 26 generally define a multi section, two-piece linear exterior shape, as best seen in FIGS. 2A-C. In some embodiments, stent 20 includes a pair of circular wires 28, with a larger wire being located at apex 26g, and a second, smaller circular wire 28 being located proximate to the attachment of the skirt to distal end 20a of the cylindrical body.

In various embodiments, the wire stent matrix 22 of stent 20 may be connected to the graft material 24 by a plurality of stitched connections. The stitched connections are not particularly limited and may include any stitched connections and/or methods known in the art. With regards to wire supports 26f, these wires may be stitched to graft material 24 along the axial length of pleat 26c. Circular support wire 28 is stitched to the shoulder segment 26e of skirt 26. In some embodiments, the wire supports 26f are further coupled to circular support wire 28. However, the present disclosure also includes various embodiments that include neither circular support wire 28 or pleat wire supports 26f.

FIG. 2 is the same as FIG. 1, except with the frontal half of skirt 26 removed to show details underneath. FIG. 3 is a cross sectional view of stent 20 taken along line 3-3 of FIG. 1. In FIG. 2A, the stent 20 is shown in the same non-deployed configuration as FIG. 1. It can be seen that an extension mechanism (shown as bladder or balloon 30 that extends around the exterior of the cylindrical body of stent 20 and underneath skirt 26) may be adapted to expand the skirt 26. For example, as shown in FIGS. 2A and 2C, the stent may include bladder 30 or a series of bladders (first bladder 130 and second bladder 133 in FIG. 2C). In FIGS. 2C and 3C, the stent may comprise an inflatable expansion mechanism having a plurality of inflatable chambers when filed with fluid 34 (shown in FIG. 3C). For example, FIG. 2C shows first bladder 130 and second bladder 133 configured to expand skirt 26 when inflated. In various embodiments, the inflatable expansion mechanism having a plurality of inflatable chambers may also comprise one-way valves/channels 131. In various embodiments, a one-way valve may be desirable to help provide an additional safety feature to stent 20.

Furthermore, in various embodiments, the inclusion of multiple inflatable expansion systems may allow for the profile of skirt 26 to be reduced. Without being limited to any theory, it is believe that reducing the profile of skirt 26 may allow for the cylindrical body of stent 20 to be wider and, thus, allow more blood to flow through stent 20.

In certain embodiments, bladder 30 is fabricated from an elastic material. Bladder 30 defines a closed interior that is filled with a fluid 34, such as a liquid. As shown in FIG. 2A, bladder 30 is in a relaxed state with little or no pressure differential between the interior of the bladder and the exterior of the bladder.

As shown in FIG. 2B and 3B, the extension mechanism may be slidable along the cylindrical body of stent 20. Slidable extension mechanism 230 may be pivotally connected to a strut or plurality of struts 233. Struts 233 may also be pivotally connected to skirt 26 (as shown with pivot point 235). Thus, as shown in FIGS. 2B and 3B, skirt 26 may expand in response to the longitudinal movement of slidable extension mechanism 230 and the pivoting of strut 233 at pivot points 231 and 235.

Furthermore, slidable extension mechanism 230 may be configured to lock in a deployed position and/or unlock from the deployed position. In various embodiments, the stent may include a detent 238 in mechanical communication with the cylindrical body. In various embodiments, the detent 238 may help secure the slidable extension mechanism 230 in a deployed position as further described below.

Also disclosed herein are stents comprising various means for expanding the skirt 26. Exemplary means for expanding skirt 26 include inflation (e.g., with a bladder 30), expansion with a plurality of struts pivotally engaged with the skirt and an extension mechanism (e.g., as illustrated in FIGS. 2B and 3B), or expansion with a plurality of struts pivotally engaged with the skirt and an extension mechanism operable with a wire/pulley.

FIGS. 3A-3C show stent 20 in various deployed configurations. With reference to FIGS. 3A and 3C, the pressure of the fluid 34 within bladder, has been increased by the surgeon. Bladder 30 in some embodiments includes a one-way valve having a release port that is in fluid communication with a lumen that may be coupled to a pressurizing means, such as a hand pump placed proximate to the handle of the stent introducer. As the surgeon pressurizes the fluid 34 in the lumen, the pressure is communicated through the one-way valve into the interior of bladder 30. This causes bladder 30 to expand to a stressed state, the shape of which is dependent upon the pressure differential across the bladder walls. In so pressurizing, the inner diameter of bladder 30 presses against the outer diameter of the cylindrical body of stent 20. An upper and outer portion of bladder 30 presses against the inner walls of skirt 26. Preferably, the bladder 30 contacts the wire supports 26f of skirt 26.

This expansion of bladder 30 results in the movement of skirt 26 relative to the cylindrical body of stent 20. Since skirt 26 is attached to the distal end 20a, this expansion causes the opened end 26a of the skirt to move both distally (i.e., toward end 20a), and further outwardly (axially), pivoting about the point of attachment of the skirt to the distal end 20a of the cylindrical body. Referring to FIG. 3, a skirt 26 in the deployed state can have one or more conical surfaces, such as a first, short conical surface extending from the attachment of the skirt to the body at the apex 26g, and a second, longer conical section extending from apex 26g to the free distal end 26a of the skirt.

So as to evenly distribute the inwardly pressing load of bladder 30 onto the wire stent matrix 22 forming the cylindrical body, some embodiments include a substantially circular inner support wire 29 (as shown in FIG. 3A) that is placed so as to evenly distribute the compression loads into the cylindrical body, and further to discourage any bulging of graft material that would otherwise be unsupported by wire. In some embodiments, inner support wire 29 is attached by a plurality of stitched connections to the graft material proximate to distal end 20a, and in yet other embodiments, wire 29 is also coupled to bladder 30 by threads that surround both the wire and the bladder and pass through the graft wall.

FIG. 3 shows that in some embodiments, bladder 30 presses upon a portion of the skirt 26 that is below and outward of the shoulder segment 26e. In so doing, the inflation of bladder 30 results in an uplifting of circular support 28. In yet other embodiments, in which there is a modified geometric relationship between the shoulder 26 and the location at which the bladder 30 presses against skirt 26, the apex 26g of skirt 26 can be lifted to a position that is above (i.e., more distal than) distal end 20a.

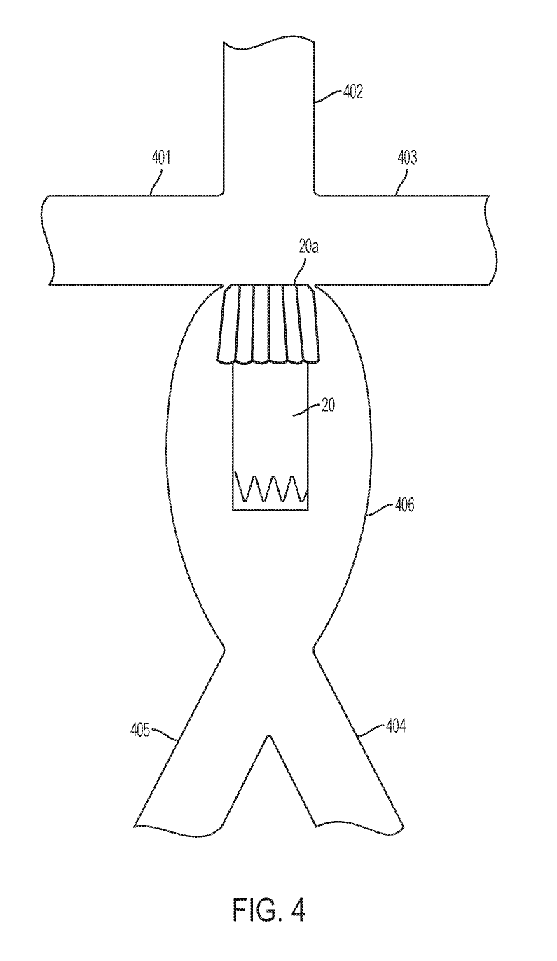

FIGS. 4-7 depict the placement of stent 20 within the descending aorta of a patient. The aneurysm 406 of the patient has substantially eliminated any neck area posterior to the renal arteries. FIG. 4 shows the stent in the non-deployed configuration. Stent 20 is located near the juncture of the left renal artery 403 and right renal artery 401. Distal end 20a is placed generally at the lower side of the lumens of the renal arteries.

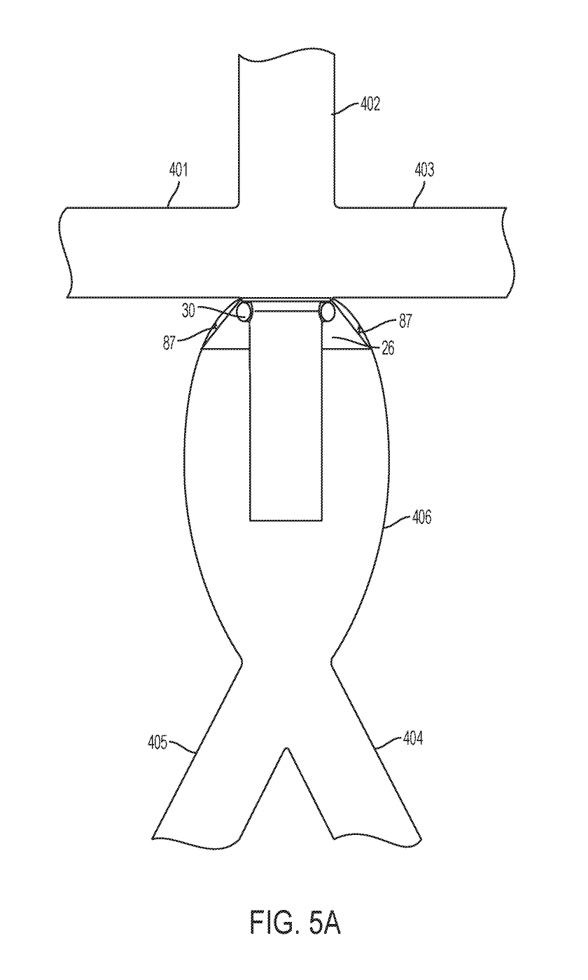

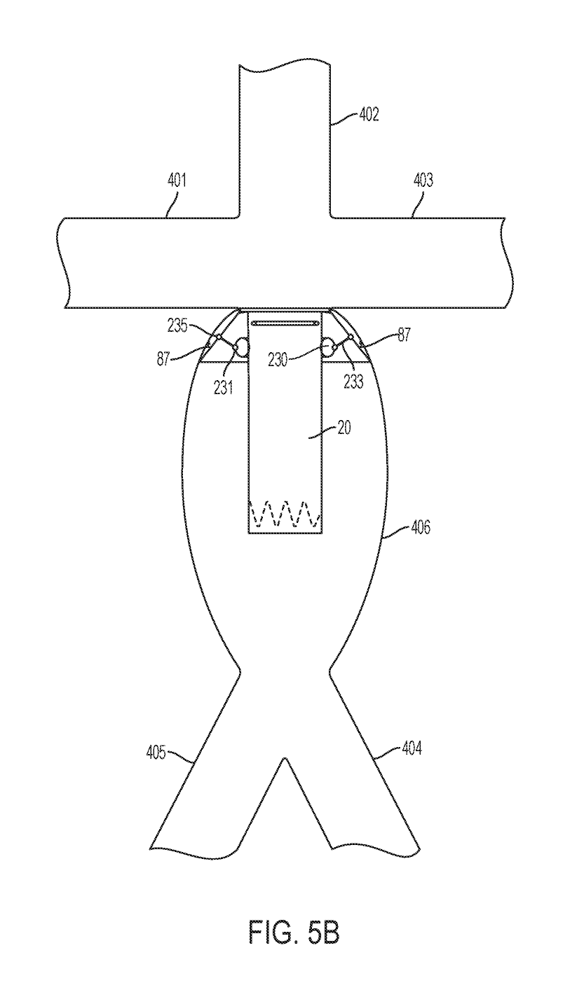

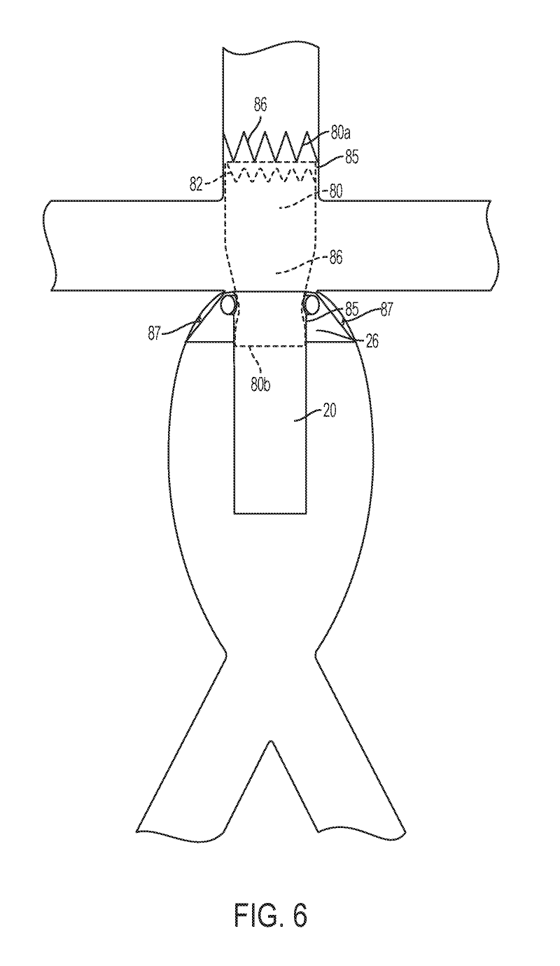

FIGS. 5A and 5B show stent 20 in the deployed configuration. FIG. 5A shows stent 20 where bladder 30 has been inflated so as to bring the skirt 26 into substantial contact with the walls of the aneurysm, while FIG. 5B illustrates stent 20 with slidable expansion mechanism 230 and struts 233. In some embodiments, stent 20 is adapted and configured such that apex 26g is sized and located so as to aid in the formation of a seal between the aortic lumen of the aneurysm and the graft material 24 of skirt 26. Also, as shown in FIGS. 5A and 5B, stent 20 may include barbs 87 (also shown in FIGS. 1 and 6).

Once stent 20 has been deployed into the aneurysm, a second upper arterial attachment stent 80 may deployed, as shown in FIG. 6. Stent 80 includes a central body portion not covered with graft material so as to allow the substantially free flow of blood laterally into the renal arteries. The distal end 80a of stent 80 is coupled to the aortic walls. In some embodiments, the distal end 80a of the wire stent material 82 may include barbs (not shown) for direct attachment to the lumen walls. The proximal end 80b of stent 80 is located within the distal end 20a of stent 20, thus overlapping with stent 20. Stent 80 provides frictional coupling to stent 20 and frictional and/or barbed coupling to the distal walls of the aorta.

FIG. 7 depicts the placement of a stent system 50 within the proximal end 20b of stent 20, with a main trunk 52 and branches extending into both right iliac artery 405 and left iliac artery 404. In various embodiments, stent system 50 may include a plurality of stents 20, as shown in FIG. 7. In various embodiments, the use of multiple stents 20 may help to ensure stent system 50 is sufficiently secured.

The materials of stent 20 are not particularly limited, so long as the materials are biocompatible. Exemplary materials include biocompatible organic polymers, such as polyethylene terephthalate (PET) (e.g., such as DACRON®, a registered trademark of E. I. DuPont Nemours and Company, a Delaware corporation) and polytetrafluoroethylene (PTFE), such as ePTFE, commercially available as DURAPLY®, a registered trademark of Endologix, Incorporated, a Delaware corporation.

Additionally, stent 20 may also include any biocompatible metal or metallic alloy. Exemplary metals include Nb, Fe, Cr, V, Co, Ta, Mo, W, Au, Ag, and alloys thereof In some embodiments, the metal may include a super-elastic alloy, such as nickel titanium (e.g., nitinol 55, nitinol 60, etc.). In various embodiments, the alloys may be magnetic resonance imaging (MRI) compatible or compliant (e.g., nitinol alloys). For example, second upper arterial attachment stent 80 may comprise a metal alloy, such as nitinol.

Also disclosed herein are methods of implanting a stent into a human body. The methods may include sliding a stent into a blood vessel along a guide wire. In various embodiments, the stent may be similar to stent 20 and may include a cylindrical body having a distal portion, a skirt having an apex in mechanical communication with the distal portion, and an extension mechanism adapted to expand the skirt. The method also includes deploying the skirt to secure the stent to the blood vessel, such as illustrated in FIGS. 3A-C.

As illustrated in FIGS. 3A and 3C, the deploying of the skirt may include inflating the extension mechanism. Also, in various embodiments, the skirt comprises sliding the extension mechanism along the cylindrical body, such as illustrated in FIG. 3B. In various embodiments, methods of implanting the stent may also include locking the stent in the deployed configuration, for example, by securing locking mechanism 237 with detent 238 as shown in FIGS. 2B and 3B.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. §112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A stent comprising:
a cylindrical body having a distal portion;
a skirt having an apex coupled to the distal portion; and
an extension mechanism adapted to expand the skirt.

2. The stent of claim 1, wherein the extension mechanism is inflatable.

3. The stent of claim 2, wherein the inflatable extension mechanism comprises a plurality of inflatable chambers.

4. The stent of claim 3, wherein the plurality of inflatable chambers are interconnected.

5. The stent of claim 4, wherein the plurality of inflatable chambers are interconnected with intermediary chambers comprising a one-way valve.

6. The stent of claim 1, wherein the extension mechanism is slidable along the cylindrical body.

7. The stent of claim 6, wherein the extension mechanism is configured to lock in a deployed position.

8. The stent of claim 7, wherein the extension mechanism is configured to unlock from the deployed position.

9. The stent of claim 8, wherein the stent comprises a detent in mechanical communication with the cylindrical body.

10. The stent of claim 1, wherein the skirt comprises a wire support.

11. The stent of claim 1, wherein the skirt is pleated.

12. The stent of claim 1, wherein the skirt comprises a plurality of barbs.

13. The stent of claim 6, wherein the extension mechanism comprises a plurality of struts pivotally connected to the skirt and a rib.

14. A stent comprising:
a cylindrical body having a distal portion;
a skirt having an apex coupled to the distal portion;
means for expanding the skirt.

15. The stent of claim 14, wherein the expanding means includes an inflatable member.

16. The stent of claim 14, wherein the expanding means includes a plurality of struts pivotally engaged with the skirt and an extension mechanism.

17. A method of implanting a stent into a human body comprising
sliding a stent into a blood vessel along a guide wire, the stent comprising
a cylindrical body having a distal portion;
a skirt having an apex coupled to the distal portion; and
an extension mechanism adapted to expand the skirt; and
expanding the skirt to secure the stent to the blood vessel.

18. The method of claim 17, wherein expanding the skirt comprises inflating the extension mechanism.

19. The method of claim 17, wherein expanding the skirt comprises sliding the extension mechanism along the cylindrical body.

20. The method of claim 17, further comprising locking the stent in a deployed configuration.
